(19) United States
(12) Patent Application Publication
(10) Pub. No.: US 2022/0370732 A1
(43) Pub. Date: Nov. 24, 2022

(54) PUNCTURE TOOL AND CATHETER ASSEMBLY

(71) Applicant: Murata Manufacturing Co., Ltd., Nagaokakyo-shi (JP)

(72) Inventors: Katsuhisa HIGASHIYAMA, Nagaokakyo-shi (JP); Tatsuya NAMATSU, Nagaokakyo-shi (JP); Keisei SOWA, Nagaokakyo-shi (JP); Koji TANAKA, Nagaokakyo-shi (JP); Takumi ONO, Nagaokakyo-shi (JP)

(21) Appl. No.: 17/747,330

(22) Filed: May 18, 2022

(30) Foreign Application Priority Data
May 18, 2021 (JP) 2021-083684
Mar. 22, 2022 (JP) 2022-045046

(51) Int. Cl. A61M 5/42; A61M 25/01

(52) U.S. Cl. CPC A61M 5/427; A61M 25/0105; A61M 2230/04; A61M 2205/3331; A61M 2205/3317; A61M 2205/3306; A61M 2025/0166

(57) ABSTRACT

A catheter assembly is provided that includes a puncture tool and a catheter. The puncture tool includes a hollow puncture needle configured to puncture a blood vessel, a detection portion that protrudes forward from a tip of the puncture needle and detects pulsation of the blood vessel, and a determination portion that determines whether the blood vessel is a vein or an artery based on the pulsation of the blood vessel detected by the detection portion. The detection portion includes a probe provided inside the hollow puncture needle that is configured to advance and retreat in an axial direction, and a sensor that detects pulsation of a blood vessel transmitted via the probe.

### CROSS REFERENCE TO RELATED APPLICATIONS

The present application claims priority to Japanese Patent Application No. 2021-083684, filed May 18, 2021, and to Japanese Patent Application No. 2022-045046, filed Mar. 22, 2022, the entire contents of each of which are hereby incorporated by reference.

### FIELD OF THE INVENTION

The present invention relates to a puncture tool and a catheter assembly including the puncture tool.

### BACKGROUND

In a medical situation, for example, in a situation such as central vein puncture for inserting a catheter into a vein and performing infusion, puncturing a body tissue of a patient has conventionally been performed. Such puncturing (e.g., puncturing a body tissue of a patient) is usually performed while visually observing an ultrasonic tomographic image of the body tissue of the patient acquired by an ultrasonic diagnostic apparatus.

Japanese Unexamined Patent Application Publication No. 2006-175191 discloses a technique including a diffusion-type semiconductor pressure sensor that senses and responds to a pressure of a blood flow when a blood vessel is punctured with a needle tip, and detects that the blood vessel is punctured with the needle and gives notice thereof. Upon receiving the notification that the blood vessel has been punctured with the needle, an operator presses a drive button of an injector motor to start injection, and automatically injects a fixed amount of drug solution or blood.

According to the technique disclosed in Japanese Unexamined Patent Application Publication No. 2006-175191 described above, it is possible to recognize that a blood vessel is punctured without visually checking back blood (e.g., flashback) due to puncturing. However, unless the blood vessel is punctured with a needle (that is, until after the needle is inserted), it is not known whether the blood vessel into which the needle has been inserted is a vein or an artery. Therefore, it is difficult to prevent the vein and the artery from being punctured by mistake (e.g., an erroneous puncture).

### SUMMARY OF THE INVENTION

Accordingly, it is an object of the present invention to provide a puncture tool configured to determine whether a blood vessel is a vein or an artery before puncturing the blood vessel (that is, without puncturing the blood vessel) and to prevent the vein and the artery from being punctured by mistake (e.g., an erroneous puncture). Moreover, a catheter assembly including the puncture tool is also provided.

In an exemplary embodiment, a puncture tool includes a hollow puncture needle with which a blood vessel is punctured; a detection portion that is configured to protrude forward from a tip of the puncture needle and to detect pulsation of the blood vessel; and a determination portion that determines whether the blood vessel is a vein or an artery based on the pulsation of the blood vessel detected by the detection portion.

Moreover, a catheter assembly according to an exemplary aspect includes the puncture tool; and a catheter that internally contacts an outer peripheral surface of the puncture tool and accommodates the puncture tool so it is configured to be slidable in an axial direction.

According to the puncture tool or the catheter assembly of an exemplary aspect, the pulsation of the blood vessel can be detected by causing the detection portion to protrude forward from the tip of the puncture needle before the puncture needle is inserted into the blood vessel (i.e., before puncturing). Based on the detected pulsation of the blood vessel, it can be determined whether the blood vessel is a vein or an artery. Therefore, before the puncture needle is inserted into the blood vessel, it is possible to determine whether the blood vessel is a vein or an artery. As a result, the exemplary embodiment can prevent the vein and the artery from being punctured by mistake (e.g., erroneous puncture).

According to the exemplary embodiment of the present invention, it is possible to determine whether a blood vessel is a vein or an artery before puncturing the blood vessel (that is, without puncturing the blood vessel), and it is possible to prevent the vein and the artery from being punctured by mistake (erroneous puncture).

### BRIEF DESCRIPTION OF THE DRAWINGS

FIG. 1 is a perspective view illustrating a puncture tool according to an exemplary embodiment and a catheter assembly including the puncture tool;

FIG. 2 is a schematic view illustrating a configuration of a detection portion forming the puncture tool according to the exemplary embodiment;

FIG. 3 is a schematic view illustrating another example of the detection portion forming the puncture tool according to the exemplary embodiment;

FIG. 4A and FIG. 4B are diagrams illustrating states of the puncture tool according to the exemplary embodiment before retraction and after retraction, respectively;

FIG. 5 is a diagram illustrating an example of a notification portion forming the puncture tool according to the exemplary embodiment;

FIG. 6 is a graph illustrating impedance characteristics (peak shift) of a sensor before contact with a blood vessel and after contact with a blood vessel;

FIG. 7 is a graph illustrating an amplitude of pulsation (pressure) of each of a vein and an artery;

FIG. 8 is a diagram (part 1) illustrating a puncture method (puncture procedure) using a catheter assembly according to the exemplary embodiment;

FIG. 9 is a diagram (part 2) illustrating the puncture method (puncture procedure) using the catheter assembly according to the exemplary embodiment;

FIG. 10 is a diagram (part 3) illustrating the puncture method (puncture procedure) using the catheter assembly according to the exemplary embodiment;

FIG. 11 is a diagram (part 4) illustrating the puncture method (puncture procedure) using the catheter assembly according to the exemplary embodiment; and

FIG. 12 is a diagram (part 5) illustrating the puncture method (puncture procedure) using the catheter assembly according to the exemplary embodiment.

### DETAILED DESCRIPTION OF EMBODIMENTS

Hereinafter, an exemplary embodiment of the present invention will be described in detail with reference to the drawings. Note that in the drawings, the same or corresponding portions are denoted by the same reference numerals. In addition, in the drawings, the same elements are denoted by the same reference numerals, and redundant description will be omitted.

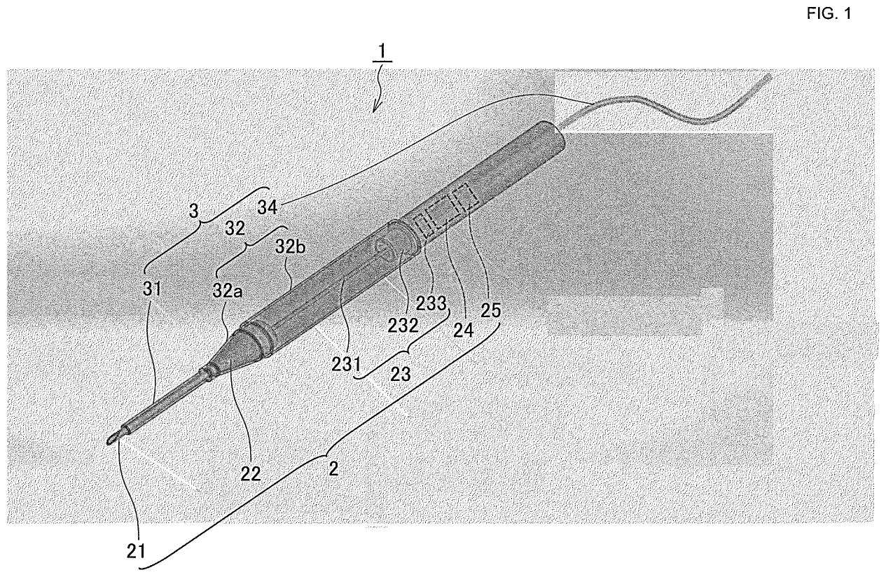

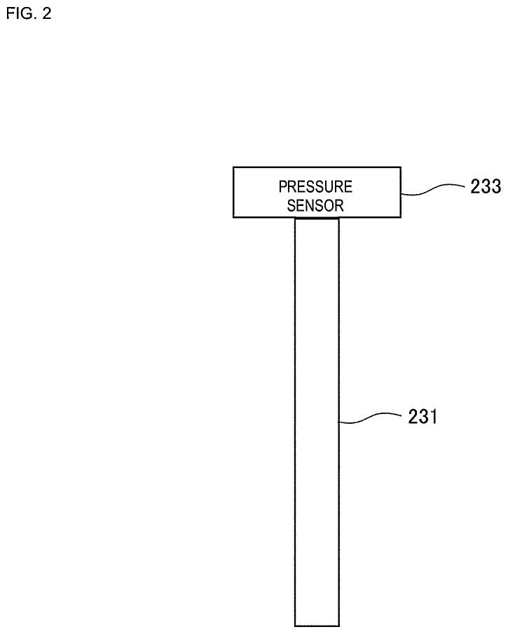

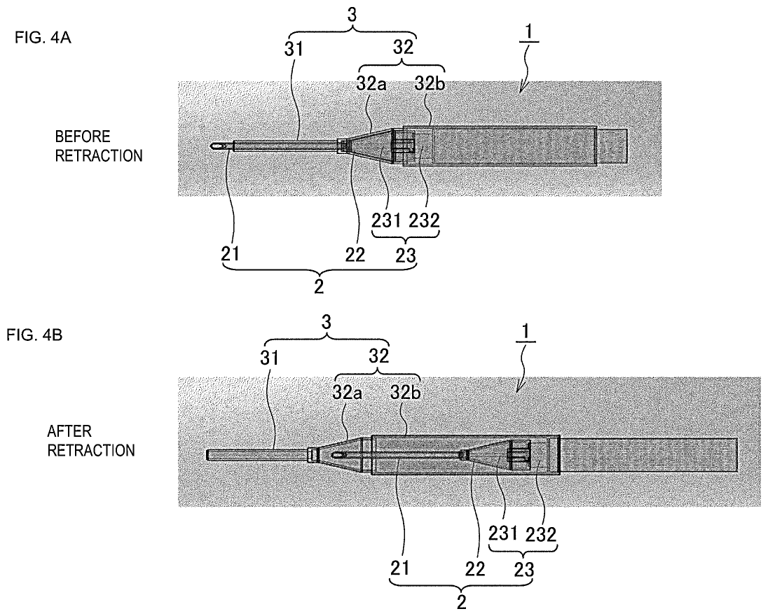

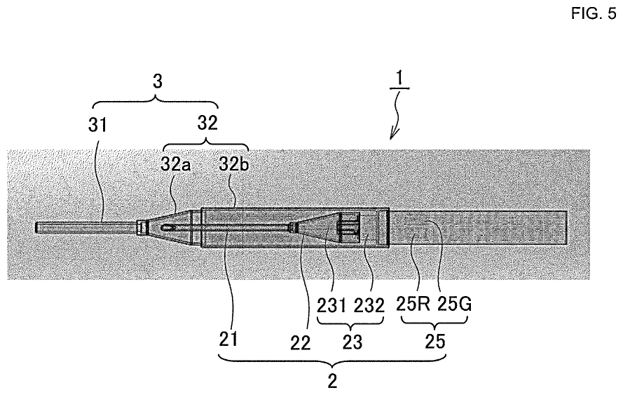

First, the configurations of a puncture tool 2 according to an exemplary embodiment and a catheter assembly 1 including the puncture tool 2 will be described with reference to FIGS. 1 to 2 and 4 to 5. FIG. 1 is a perspective view illustrating the puncture tool 2 and the catheter assembly 1 including the puncture tool 2. It is noted that FIG. 1 illustrates a state in which a puncture needle 21 and a puncture needle hub 22 are pulled rearward. FIG. 2 is a schematic view illustrating the configuration of a detection portion 23 forming the puncture tool 2. FIG. 4A and FIG. 4B illustrate the states of the puncture tool 2 before retraction and after retraction, respectively. FIG. 5 is a diagram illustrating an example of a notification portion 25 forming the puncture tool 2.

In operation, the catheter assembly 1 including the puncture tool 2 punctures a body tissue (skin, blood vessel, or the like) and inserts a catheter 3 into, for example, a vein to perform infusion or the like.

In particular, the catheter assembly 1 including the puncture tool 2 is configured to determine whether the blood vessel is a vein or an artery before puncturing the blood vessel (that is, without puncturing the blood vessel), and thus is configured to prevent the vein and the artery from being punctured by mistake (e.g., erroneous puncture).

Therefore, the catheter assembly 1 is configured to include the puncture tool 2 and the catheter 3 that internally contacts an outer peripheral surface of the puncture tool 2 and accommodates the puncture tool 2 so as to be slidable (e.g., be relatively movable) in an axial direction. Hereinafter, each component will be described in detail.

The puncture tool 2 mainly includes the hollow puncture needle (inner needle) 21 with which a blood vessel is punctured, the detection portion 23 that is configured to protrude forward in the axial direction from a tip of the puncture needle 21 (i.e., configured to advance and retreat) and detect pulsation of the blood vessel, a determination portion 24 that determines whether the blood vessel is a vein or an artery based on the pulsation of the blood vessel detected by the detection portion 23, and the notification portion 25 that gives notice of the determination result.

In particular, the puncture needle 21 has a pointed tip, and guides a catheter needle (outer needle) 31 by puncturing the skin or the like of a patient or the like with the tip that penetrates through the catheter needle 31 and protrudes. A proximal end of the puncture needle 21 is held by the puncture needle hub (inner needle hub) 22. The puncture needle hub 22 is formed in a stepped columnar shape having a narrow tip portion that is tapered, and is arranged inside a hollow portion of a catheter hub (outer needle hub) 32 so as to be relatively movable in the axial direction.

The detection portion 23 is provided inside (i.e., inner side of) the hollow puncture needle 21 so as to be configured to advance and retreat in the axial direction (that is, capable of moving relative to the puncture needle 21). Therefore, it is possible to suppress the influence of the pressure by the body tissue at the time of puncturing and more accurately detect the pulsation of the blood vessel. In addition, since sensing can be performed at the needle tip closer to the blood vessel, pulsation can be detected with high sensitivity. When the detection portion 23 can protrude forward from the puncture needle 21 even slightly, it is possible to prevent a vein and an artery from being punctured by mistake.

More specifically, the detection portion 23 is configured to include a probe 231 provided inside (inner side of) the hollow puncture needle 21 so as to be capable of advancing and retreating (be relatively movable) in the axial direction, a probe hub 232 that holds the probe 231, and a sensor 233 that is arranged on the rear end side of the probe 231 and detects pulsation of a blood vessel transmitted via the probe 231.

In an exemplary aspect, the probe 231 is formed in an elongated rod shape having a substantially flat tip. The probe 231 is preferably formed of, for example, a metal such as stainless steel or a hard synthetic resin. In this case, the probe 231 can be moved and brought into direct contact with the blood vessel to detect the pulsation of the blood vessel, so that the pulsation of the blood vessel can be detected with higher sensitivity.

The probe hub 232 holds the probe 231 at its proximal end. The probe hub 232 is formed in a stepped cylindrical shape including a reduced-diameter portion on the tip side and an enlarged-diameter portion connected to the reduced-diameter portion. By rotating operation of the probe hub 232 in a circumferential direction, the probe 231 can be advanced and retreated in the axial direction.

The sensor 233 is arranged within the probe hub 232, for example, at the rear end of the probe 231 (see FIG. 2). Moreover, the sensor 233 is configured to detect, for example, a pressure caused by pulsation of a blood vessel. The sensor 233 is formed of, for example, a piezoelectric body (piezoelectric element) that outputs an electric signal (for example, voltage) corresponding to the pressure caused by the pulsation of a blood vessel. By using a piezoelectric body as the sensor 233, the sensor 233 can be made more compact and can be housed in the thin probe hub 232. Further, in the method of detecting the pressure, since it is sufficient to touch the blood vessel and it does not depend on the contact method, a measurement error due to an individual difference is small, and even a user having relatively low technical skills can accurately detect the pressure. On the other hand, in the method of detecting the pressure, since the pressure cannot be detected when the probe is separated from the blood vessel wall, it is possible to reduce the possibility of erroneously determining that the blood vessel is a vein.

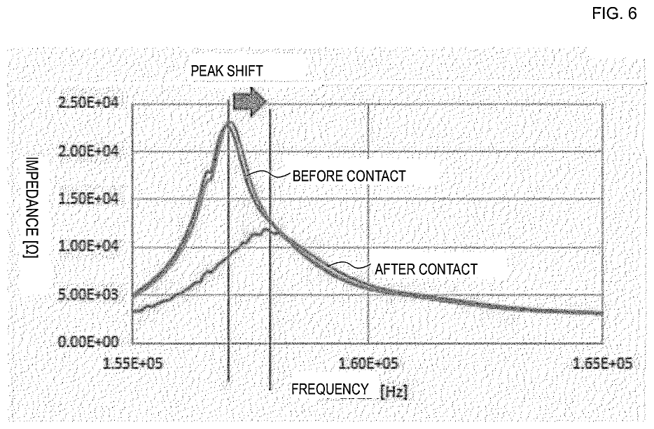

Here, FIG. 6 illustrates the impedance characteristics (peak shift) of the sensor (piezoelectric body) 233 before contact with the blood vessel and after contact with the blood vessel. It is noted that in FIG. 6, the horizontal axis represents frequency (Hz), and the vertical axis represents impedance (Q) of the sensor (piezoelectric body) 233. As illustrated in FIG. 6, the peak value of the impedance decreases and the peak frequency of the impedance shifts to a higher frequency side (peak shift) after contact with the blood vessel compared to before contact with the blood vessel. Therefore, by detecting the peak shift or impedance difference of the impedance of the sensor (piezoelectric body) 233, it is possible to determine whether or not the probe 231 has contacted the blood vessel. It is noted that the electric signal detected by the sensor 233 is output to the determination portion 24.

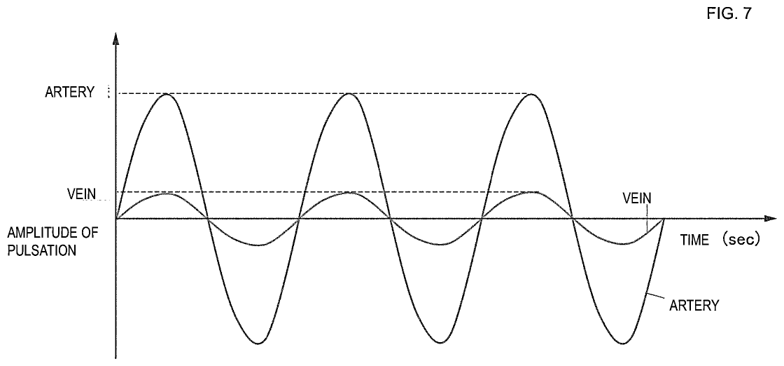

The determination portion 24 is configured to determine whether the blood vessel is a vein or an artery based on, for example, the magnitude of the amplitude (i.e., the pressure) of the pulsation of the blood vessel detected by the sensor 233. Here, FIG. 7 illustrates the amplitude of pulsation of each of the vein and the artery. In FIG. 7, the horizontal axis represents time, and the vertical axis represents the amplitude of pulsation. As illustrated in FIG. 7, the amplitude of the artery is larger than the amplitude of the vein. Therefore, when the detection value of the sensor 233 is less than a predetermined threshold value set between a minimum value of the amplitude of the artery and a maximum value of the amplitude of the vein, for example, the determination portion 24 determines that the blood vessel in contact is the vein. On the other hand, when the detection value of the sensor 233 is equal to or greater than the predetermined threshold value, the determination portion 24 determines that the blood vessel in contact is an artery. It is noted that an amount of the peak shift of the impedance of the sensor (e.g., the piezoelectric body) 233 described above increases as the pulsation increases. Therefore, the amount of the peak shift of the artery is larger than that of the vein. Therefore, it may be determined whether it is an artery or a vein based on the amount of the peak shift of the impedance. In addition, the determination of whether it is an artery or a vein includes not only direct determination, but also indirect determination, such as determination that the amplitude of a detected signal is equal to or greater than/less than a threshold value and determination that the period (peak shift amount) of a detected signal is equal to or greater than/less than a threshold value.

In addition, when the same determination result (e.g., determination result of whether it is a vein or an artery) is obtained a predetermined number of times or more (for example, twice or more consecutively), the determination portion 24 confirms the determination result. In this way, the influence of pulse-like noise or the like can be eliminated, and more accurate determination can be performed (e.g., erroneous determination can be prevented). It is also noted that the determination portion 24 can be configured by a CPU that executes calculation and a memory that stores a program and data (configured by software) or can be configured by a comparator including such as an operational amplifier (configured by hardware) according to various exemplary aspects. The determination result by the determination portion 24 is output to the notification portion 25.

The notification portion 25 notifies a user of the determination result of whether the blood vessel is a vein or an artery. As illustrated in FIG. 5, the notification portion 25 is configured to include, for example, a red LED25R indicating that the blood vessel is an artery and a green LED25G indicating that the blood vessel is a vein. When it is determined that the blood vessel is an artery, the notification portion 25 turns on the red LED25R. On the other hand, when it is determined that the blood vessel is a vein, the notification portion 25 turns on the green LED25G. It should be appreciated that the colored LEDs are an exemplary aspect and that a notification of the determination result may be given by sound, character display, or the like instead of light emission (LED), as would be appreciated to one skilled in the art. As such, this allows the user to accurately recognize whether the blood vessel in contact with the catheter assembly 1 is an artery or a vein.

The catheter 3 is configured to mainly include the catheter needle (outer needle) 31, the catheter hub (outer needle hub) 32 that holds the catheter needle 31, and an infusion tube 34. The catheter 3 internally contacts the outer peripheral surface of the puncture tool 2 and accommodates the puncture tool 2 so as to be slidable (be relatively movable) in the axial direction.

The catheter needle 31 is a flexible tubular member having a small diameter, and is formed to have a length that can be introduced into and indwelled in the body of a patient. Inside the catheter needle 31, a lumen is formed penetrating along the direction of axis. This lumen has an inner diameter through which the puncture needle 21 can be inserted. In exemplary aspects, the catheter needle 31 may be formed by a resin material, and more particularly, a soft resin material, may be suitable.

In an initial state (state before retraction), the puncture needle (inner needle) 21 is arranged inside the catheter needle (outer needle) 31 in a state where the tip of the puncture needle (inner needle) 21 protrudes. Further, as described above, the probe 231 is arranged inside the puncture needle 21.

The catheter hub 32 is a stepped hollow cylindrical member that holds the proximal end of the catheter needle 31, and is configured to include a tapered portion 32a formed in a tapered shape and a cylindrical portion 32b formed in a cylindrical shape. The catheter hub 32 is preferably made of a transparent resin so that the inside of all or part of the catheter hub 32 can be visually checked by a user, for example.

As illustrated in FIG. 4B, the catheter hub 32 is configured such that the probe 231 and the puncture needle 21 (puncture tool 2) can be retracted (retreated) and housed. That is, the catheter hub 32 is configured to function as a retraction portion according to the present disclosure. It is also noted that FIG. 4B is a diagram illustrating a state in which the puncture needle 21 and the probe 231 are retracted to the catheter hub 32. In this way, by retracting the puncture needle 21 and the probe 231 to the catheter hub 32, it is possible to prevent the flow of a drug solution or blood from being hindered by the probe 231 or the like such as when injecting the drug solution or collecting blood.

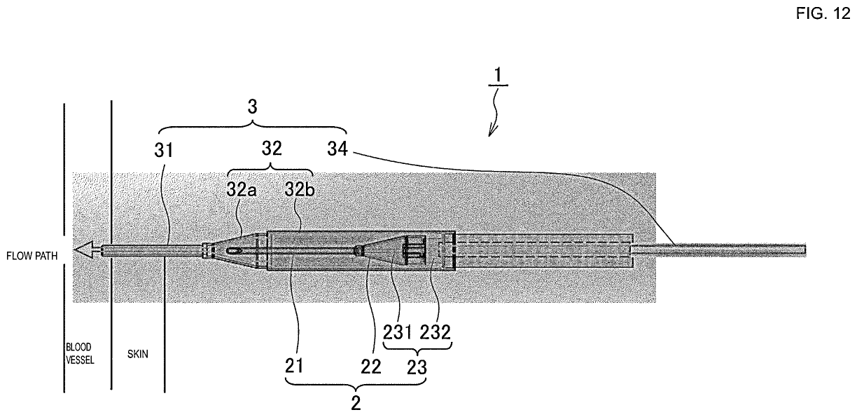

As illustrated in FIG. 12, the infusion tube 34 passes through the inside (i.e., the hollow portion) of the probe hub 232 and is connected to the catheter hub 32 (i.e., the cylindrical portion 32b) in a liquid-tight manner. Therefore, for example, a drug solution or the like can be supplied to a vein of a patient through the catheter 3 including the infusion tube 34.

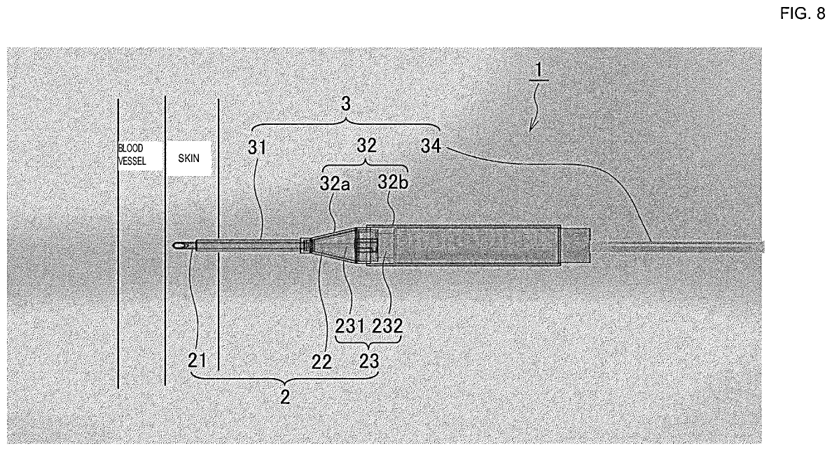

Next, a method of using the catheter assembly 1 (e.g., a procedure of inserting the catheter assembly 1 into a vein of a patient) will be described with reference to FIGS. 8 to 12. FIGS. 8 to 12 are diagrams (parts 1 to 5) illustrating a puncture method (e.g., a puncture procedure) using the catheter assembly 1.

First, as illustrated in FIG. 8, a user, such as a doctor, punctures the skin of a patient with the catheter assembly 1 (puncture tool 2, catheter 3) while visually observing an ultrasonic tomographic image of the body tissue of the patient, for example, and advances the tip of the catheter assembly 1 to the front of a blood vessel. At this time, the puncture needle 21 is arranged inside the catheter needle 31 in an overlapping manner in a state in which the tip of the puncture needle 21 is protruded. In addition, the probe 231 is arranged inside the puncture needle 21 (i.e., arranged in a state in which the tip thereof is not protruded).

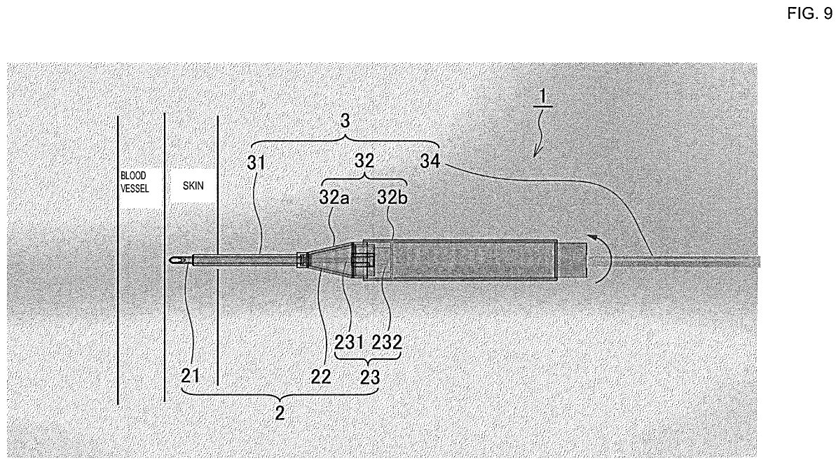

Next, as illustrated in FIG. 9, the user, such as a doctor, rotates (e.g., rotationally operates) the probe hub 232 about the axis to release an engagement state between the probe 231 and the probe hub 232 holding the probe 231, and the puncture needle 21 and the puncture needle hub 22 holding the puncture needle 21, and causes the probe 231 to protrude forward from the tip of the puncture needle 21.

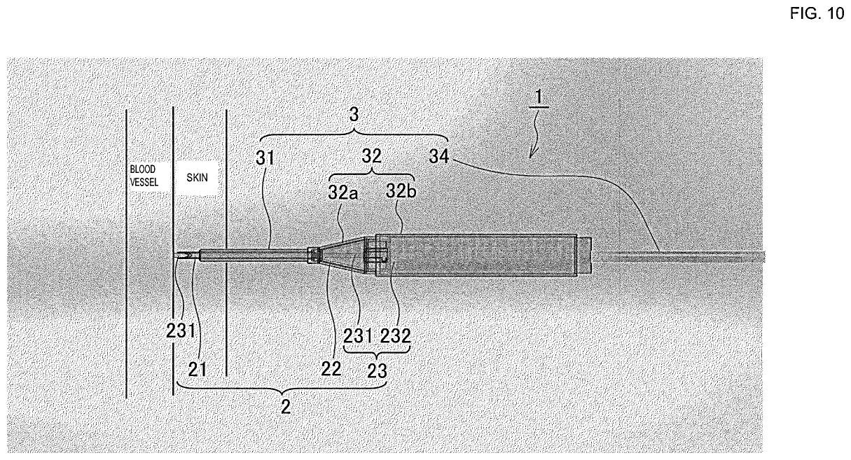

Next, as illustrated in FIG. 10, the user, such as a doctor, causes the probe 231 to hit the blood vessel wall, and determines whether the blood vessel is a vein or an artery. It is noted that since the method of determining the vein and the artery is as described above, a detailed description thereof will be omitted here.

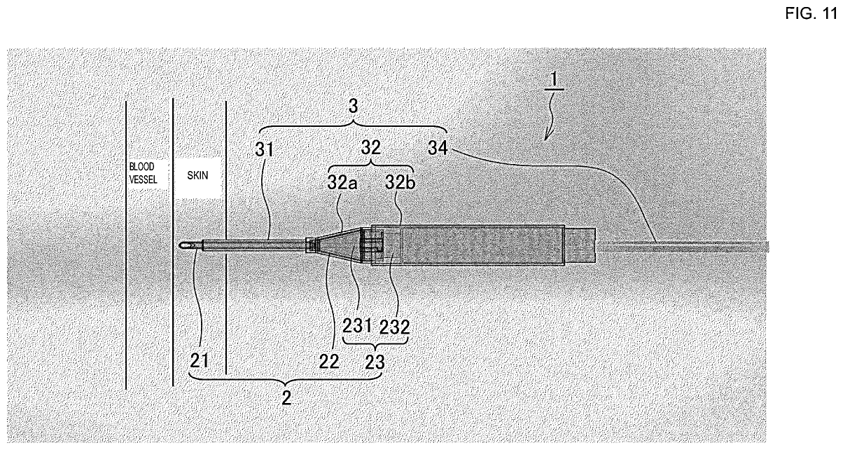

When it is determined that the blood vessel is a vein (when the determination is confirmed), as illustrated in FIG. 11, the user pulls (e.g., retreats) the probe hub 232 and changes the tip from the probe 231 to the puncture needle 21. Then, the puncture needle 21 and the catheter needle 31 are inserted into the vein in an overlapped state (venipuncture).

Next, as illustrated in FIG. 12, the user, such as a doctor, retreats the probe 231 and the puncture needle 21 to be retracted into the catheter hub 32 (cylindrical portion 32b). As a result, the catheter 3 is inserted into the vein, and an infusion line is constructed. Thereafter, the user supplies, for example, a drug solution or the like to the vein of the patient through the catheter 3 including the infusion tube 34.

As described above in detail, according to the present embodiment, for example, before the puncture needle 21 is inserted into the blood vessel (before puncturing), the detection portion 23 (i.e., probe 231) protrudes forward from the tip of the puncture needle 21, and thus the pulsation of the blood vessel can be detected. Based on the detected pulsation of the blood vessel, it can be determined whether the blood vessel is a vein or an artery. Therefore, before the puncture needle 21 is inserted into the blood vessel, it is possible to determine whether the blood vessel is a vein or an artery. Here, it should be appreciated that detecting pulsation also includes the possibility that the detecting may be that there is no pulsation. It is also noted that the absence of pulsation includes not only a case where there is no pulsation, but also a case where a peak of a detected signal is less than a predetermined threshold value.

As a result, it is possible to determine whether the blood vessel is a vein or an artery before puncturing the blood vessel (that is, without puncturing the blood vessel), and it is possible to prevent the vein and the artery from being punctured by mistake (e.g., erroneous puncture). In addition, since it is automatically determined whether the blood vessel is a vein or an artery, the work load of the user can be reduced. Further, even a user with low skills can accurately perform puncturing.

In general, it is noted that although the exemplary embodiment of the present invention has been described above, the present invention is not limited to the above-described embodiment and various modifications are possible. For example, although a piezoelectric body is used as the sensor 233 in the above-described embodiment, a diaphragm-type pressure sensor, for example, may be used instead of the piezoelectric body according to an alternative aspect.

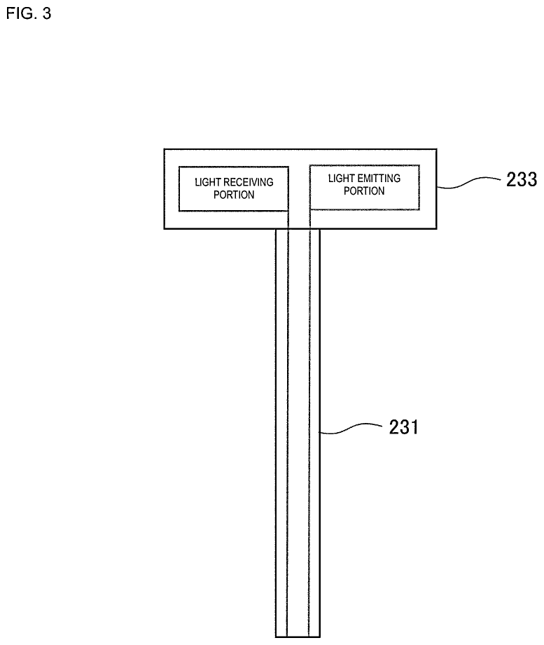

Additionally, instead of the piezoelectric body, for example, an optical fiber sensor configured to detect pulsation from an optical path difference that changes in accordance with the pulsation (e.g., vibration) may be used. In addition, instead of the piezoelectric body, for example, a Doppler sensor that configured to detect pulsation based on a difference in time in which waves such as ultrasonic waves, millimeter waves, infrared rays, and light come and go may be used (see FIG. 3). Further, instead of the piezoelectric body, an acceleration sensor of an electrostatic capacity type, a piezoresistive type, a thermal detection type, or the like may be used in various alternative aspects.

In the above-described exemplary embodiment, the red LED25R indicating that the blood vessel is an artery and the green LED25G indicating that the blood vessel is a vein are used as the notification portion 25, however, instead of the red LED25R and the green LED25G, for example, wireless communication such as Bluetooth® Low Energy (BLE) may be used to transmit the determination result to the outside and give notice of (e.g., display) the determination result.

It is also noted that the materials, manufacturing methods, and the like of the above-described components that may be the same as those of known medical catheters of the same type, for example, synthetic resin molded products by injection molding, metal products by various manufacturing methods, and the like can be used.

What is claimed is:

1. A puncture tool comprising:
a hollow puncture needle configured to puncture a blood vessel;
a detection portion configured to protrude forward from a tip of the puncture needle and configured to detect a pulsation of the blood vessel; and
a determination portion configured to determine whether the blood vessel is a vein or an artery based on the pulsation of the blood vessel detected by the detection portion.

2. The puncture tool according to claim 1, wherein the detection portion is configured to be movable inside the hollow puncture needle.

3. The puncture tool according to claim 1, wherein the detection portion includes:
a probe that is configured to be movable inside the hollow puncture needle; and
a sensor that is arranged on a rear end side of the probe opposite the tip of the puncture needle and that is configured to detect the pulsation of the blood vessel transmitted via the probe.

4. The puncture tool according to claim 3, wherein the determination portion is configured to determine whether the blood vessel is a vein or an artery based on an amplitude of the pulsation of the blood vessel.

5. The puncture tool according to claim 4, wherein the determination portion is configured to confirm a determination of the blood vessel when the determination portion determines a same determination result for a predetermined number of times.

6. The puncture tool according to claim 4, wherein the determination portion determines that the blood vessel is a vein when the amplitude of the pulsation of the blood vessel is less than a predetermined threshold value.

7. The puncture tool according to claim 6, wherein the determination portion determines that the blood vessel is an artery when the amplitude of the pulsation of the blood vessel is greater than the predetermined threshold value.

8. The puncture tool according to claim 3, wherein the sensor is configured to detect a pressure caused by the pulsation of the blood vessel.

9. The puncture tool according to claim 8, wherein the sensor comprises a piezoelectric body that is configured to output an electric signal that corresponds to the pressure caused by the pulsation of the blood vessel.

10. The puncture tool according to claim 1, further comprising a notification portion configured to provide a notice of a determination result generated by the determination portion of whether the blood vessel is a vein or an artery.

11. The puncture tool according to claim 1, further comprising a notification portion configured to provide a first notification when the determination portion determines that the blood vessel is an artery.

12. The puncture tool according to claim 1, wherein the hollow puncture needle comprises a proximal end held by a puncture needle hub having a stepped columnar shape with a tapered tip.

13. The puncture tool according to claim 1, wherein the determination portion comprises a computer processing unit configured to execute a software program stored on memory for determining whether the blood vessel is a vein or an artery based on the detected pulsation of the blood vessel.

14. The puncture tool according to claim 1, wherein the determination portion comprises a hardware configuration including a comparator for determining whether the blood vessel is a vein or an artery by comparing a value of the detected pulsation of the blood vessel to a predetermined threshold value.

15. A puncture tool comprising:
a hollow puncture needle configured to puncture a blood vessel;
a detection portion that includes a probe configured to advance in an axial direction from the hollow puncture needle and a sensor that is configured to detect a pulsation of the blood vessel; and
a determination portion configured to compare a value of the detected pulsation to a threshold value to determine whether the blood vessel is a vein or an artery based on a comparison result.

16. The puncture tool according to claim 15, wherein the sensor is configured to detect a pressure caused by the detected pulsation of the blood vessel.

17. The puncture tool according to claim 16, wherein the sensor comprises a piezoelectric body that is configured to output an electric signal that corresponds to the pressure caused by the detected pulsation of the blood vessel.

18. The puncture tool according to claim 15, wherein the determination portion is configured to determine whether the blood vessel is a vein or an artery based on an amplitude of the detected pulsation of the blood vessel.

19. A catheter assembly comprising:
the puncture tool according to claim 1; and
a catheter that internally contacts an outer peripheral surface of the puncture tool and accommodates the puncture tool so as to be slidable.

20. The catheter assembly according to claim 19, further comprising at least a retraction portion that is configured to retract a probe movably provided inside the puncture needle from the puncture needle and houses the probe.
